(19) United States
(12) Patent Application Publication
(10) Pub. No.: US 2013/0178449 A1
(43) Pub. Date: Jul. 11, 2013

(54) 2-Methylene-20(21)-Dehydro-19,24,25,26,27-Pentanor-Vitamin D Analogs

(71) Applicants: Hector F. DeLuca, Deerfield, WI (US); Margaret Clagett-Dame, Deerfield, WI (US); Lori A. Plum, Arena, WI (US); Agnieska Glebocka, Madison, WI (US); Rafal R. Sicinski, Warsaw (PL)

(72) Inventors: Hector F. DeLuca, Deerfield, WI (US); Margaret Clagett-Dame, Deerfield, WI (US); Lori A. Plum, Arena, WI (US); Agnieska Glebocka, Madison, WI (US); Rafal R. Sicinski, Warsaw (PL)

(73) Assignee: WISCONSIN ALUMNI RESEARCH FOUNDATION, Madison, WI (US)

(21) Appl. No.: 13/547,111

(22) Filed: Jul. 12, 2012

Related U.S. Application Data
(60) Provisional application No. 61/508,882, filed on Jul. 18, 2011.

(51) Int. Cl. C07C 401/00

(52) U.S. Cl. CPC C07C 401/00
USPC 514/167; 552/653

(57) ABSTRACT

This invention discloses 2-methylene-20(21)-dehydro-19,24,25,26,27-pentanor-vitamin D analogs, and specifically 2-methylene-20(21)-dehydro-19,24,25,26,27-pentanor-1α-hydroxyvitamin D₃, and pharmaceutical uses therefor. This compound exhibits relatively high transcription activity as well as pronounced activity in arresting the proliferation of undifferentiated cells and inducing their differentiation to the monocyte thus evidencing use as an anti-cancer agent and for the treatment of skin diseases such as psoriasis as well as skin conditions such as wrinkles, slack skin, dry skin and insufficient sebum secretion. This compound also shows lower activity in vivo on bone calcium mobilization and lower in vivo intestinal calcium transport activity as compared to the native hormone 1α,25-dihydroxyvitamin D₃, and therefore may be used to treat autoimmune disorders or inflammatory diseases in humans as well as secondary hyperparathyroidism and renal osteodystrophy. This compound may also be used for the treatment or prevention of obesity.

### BACKGROUND OF THE INVENTION

This invention relates to vitamin D compounds, and more particularly to 2-methylene-20(21)-dehydro-19,24,25,26,27-pentanor-vitamin D analogs and their pharmaceutical uses, and specifically to 2-methylene-20(21)-dehydro-1α-hydroxy-19,24,25,26,27-pentanor-vitamin D₃ and its pharmaceutical uses.

The natural hormone, 1α,25-dihydroxyvitamin D₃ and its analog in ergosterol series, i.e. 1α,25-dihydroxyvitamin D₂ are known to be highly potent regulators of calcium homeostasis in animals and humans, and their activity in cellular differentiation has also been established, Ostrem et al., Proc. Natl. Acad. Sci. USA, 84, 2610 (1987). Many structural analogs of these metabolites have been prepared and tested, including 1α-hydroxyvitamin D₃, 1α-hydroxyvitamin D₂, various side chain homologated vitamins and fluorinated analogs. Some of these compounds exhibit an interesting separation of activities in cell differentiation and calcium regulation. This difference in activity may be useful in the treatment of a variety of diseases such as renal osteodystrophy, vitamin D-resistant rickets, osteoporosis, psoriasis, and certain malignancies.

Another class of vitamin D analogs, i.e. the so called 19-nor-vitamin D compounds, is characterized by the replacement of the A-ring exocyclic methylene group (carbon 19), typical of the vitamin D system, by two hydrogen atoms. Biological testing of some 19-nor-analogs (e.g., 1α,25-dihydroxy-19-nor-vitamin D₃) revealed a selective activity profile with high potency in inducing cellular differentiation, and reduced calcium mobilizing activity. Thus, these compounds are potentially useful as therapeutic agents for the treatment of malignancies, or the treatment of various skin disorders. Two different methods of synthesis of such 19-nor-vitamin D analogs have been described (Perlman et al., Tetrahedron Lett. 31, 1823 (1990); Perlman et al., Tetrahedron Lett. 32, 7663 (1991), and DeLuca et al., U.S. Pat. No. 5,086,191).

In U.S. Pat. No. 4,666,634, 2β-hydroxy and alkoxy (e.g., ED-71) analogs of 1α,25-dihydroxyvitamin D₃ have been described and examined as potential drugs for osteoporosis and as antitumor agents. See also Okano et al., Biochem. Biophys. Res. Commun. 163, 1444 (1989). Other 2-substituted (with hydroxyalkyl, e.g., ED-120, and fluoroalkyl groups) A-ring analogs of 1α,25-dihydroxyvitamin D₃ have also been prepared and tested (Miyamoto et al., Chem. Pharm. Bull. 41, 1111 (1993); Nishii et al., Osteoporosis Int. Suppl. 1, 190 (1993); Posner et al., J. Org. Chem. 59, 7855 (1994), and J. Org. Chem. 60, 4617 (1995)).

2-substituted analogs of 1α,25-dihydroxy-19-nor-vitamin D₃ have also been synthesized, i.e. compounds substituted at 2-position with hydroxy or alkoxy groups (DeLuca et al., U.S. Pat. No. 5,536,713), with 2-alkyl groups (DeLuca et at U.S. Pat. No. 5,945,410), and with 2-alkylidene groups (DeLuca et al U.S. Pat. No. 5,843,928), which exhibit interesting and selective activity profiles. All these studies indicate that binding sites in vitamin D receptors can accommodate different substituents at C-2 in the synthesized vitamin D analogs.

In a continuing effort to explore the 19-nor class of pharmacologically important vitamin D compounds, analogs which are characterized by the presence of a methylene substituent at carbon 2 (C-2), a hydroxyl group at carbon 1 (C-1) and carbon 3 (C-3), and a shortened side chain attached to carbon 20 (C-20) have also been synthesized and tested. 1α-Hydroxy-2-methylene-19-nor-pregnacalciferol is described in U.S. Pat. No. 6,566,352 while 1α-hydroxy-2-methylene-19-nor-homopregnacalciferol is described in U.S. Pat. No. 6,579,861 and 1α-hydroxy-2-methylene-19-nor-bishomopregnacalciferol is described in U.S. Pat. No. 6,627,622. All three of these compounds have relatively high binding activity to vitamin D receptors and relatively high cell differentiation activity, but little if any calcemic activity as compared to 1α,25-dihydroxyvitamin D₃. Their biological activities make these compounds excellent candidates for a variety of pharmaceutical uses, as set forth in the '352, '861 and '622 patents. Each of these three analogs with a truncated side chain also effectively suppresses parathyroid hormone levels, Plum et al, PNAS, 101, 6900 (2004), indicating these compounds may be useful as a therapy for suppression of secondary hyperparathyroidism caused by chronic renal failure as well as a treatment for renal osteodystrophy.

17(20)-Ene vitamin D compounds as well as vitamin D compounds having a double bond in the side chain thereof are also known, and have been proposed for various pharmacological uses. Bone diseases such as osteoporosis, skin disorders such as psoriasis, cancers such as leukemia, and cosmetic conditions such as wrinkles are just some of the applications proposed for such compounds. 17(20)-Ene compounds are described in U.S. Pat. Nos. 5,545,633; 5,929,056 and 6,399,797 while 2-alkylidene compounds having a side chain with a double bond therein are described in, for example, U.S. Pat. No. 5,843,928.

Vitamin D compounds having a double bond between positions 20 (C-20) and 21 (C-21) have also been synthesized. More specifically, the compound 2-methylene-20(21)-dehydro-19-nor-1α,25-dihydroxyvitamin D₃, and various pharmacological uses thereof, are described in U.S. Pat. No. 7,888,339.

### SUMMARY OF THE INVENTION

The present invention is directed toward 2-methylene-20(21)-dehydro-19,24,25,26,27-pentanor-vitamin D analogs, their biological activity, and various pharmaceutical uses for these compounds. These new vitamin D compounds not known heretofore are the 19-nor-vitamin D analogs (i.e. vitamin D compounds having the A-ring exocyclic methylene group at carbon-10 removed and replaced with two hydrogen atoms) having a methylene group at the 2-position (C-2) of the A-ring, and a 1-methylene-propyl group substituted at carbon-17, such that a double bond is located between carbon atoms 20 and 21 in the side chain. The preferred vitamin D analog is 2-methylene-20(21)-dehydro-19,24,25,26,27-pentanor-1α-hydroxyvitamin D₃ (hereinafter referred to as “MDBE20”).

Structurally these 2-methylene-20(21)-dehydro-19-nor-vitamin D analogs are characterized by the general formula I shown below:

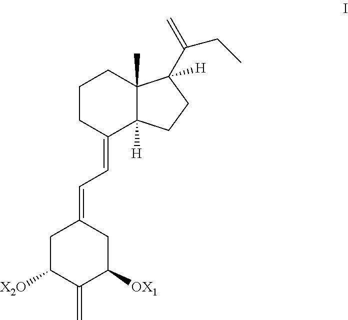

where X₁ and X₂, which may be the same or different, are each selected from hydrogen or a hydroxy-protecting group. The preferred analog is 2-methylene-20(21)-dehydro-19,24,25,26,27-pentanor-1α-hydroxyvitamin D₃ which is herein referred to as “MDBE 20,” and has the following formula Ia:

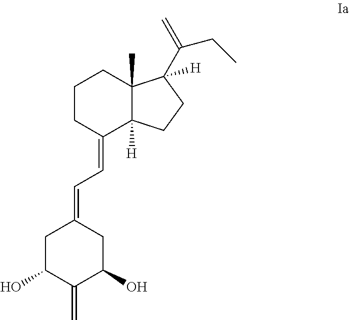 [SMILES: C=C1[C@H](O)CC(=C/C=C2\CCC[C@]3(C)[C@@H](C(=C)CC)CC[C@@H]23)C[C@H]1O]

The above compounds I, particularly Ia, exhibit a desired, and highly advantageous, pattern of biological activity. These compounds are characterized by relatively high binding to vitamin D receptors, with only slightly less affinity than that of the native hormone 1α,25-dihydroxyvitamin D₃ (See FIG. 1). These compounds also have little, if any, ability to promote intestinal calcium transport in vivo, and they would be classified as having substantially no intestinal calcium transport activity, as compared to that of 1α,25-dihydroxyvitamin D₃. These compounds I, and particularly Ia, also have little, if any, ability to mobilize calcium from bone, and they would be classified as having substantially no bone calcium mobilizing activity as compared to 1α,25-dihydroxyvitamin D₃.

It is undesirable to raise serum calcium to supraphysiologic levels when suppressing the preproparathyroid hormone gene (Darwish & DeLuca, Arch. Biochem. Biophys. 365, 123-130, 1999) and parathyroid gland proliferation. These analogs having low bone calcium mobilization activity while being relatively active on cell differentiation are expected to be useful as a therapy for suppression of secondary hyperparathyroidism in subjects on dialysis having chronic renal failure, or as a treatment of renal osteodystrophy.

The compounds I, particularly Ia, of the invention have also been discovered to be especially suited for treatment and prophylaxis of human disorders which are characterized by an imbalance in the immune system, e.g. in autoimmune diseases, including multiple sclerosis, lupus, diabetes mellitus, host versus graft rejection, and rejection of organ transplants; and additionally for the treatment of inflammatory diseases, such as rheumatoid arthritis, asthma, and inflammatory bowel diseases such as celiac disease, ulcerative colitis and Crohn's disease.

The above compounds I, and particularly Ia, are also characterized by relatively high cell differentiation activity and in promoting transcription. Thus, these compounds also provide a therapeutic agent for the treatment of psoriasis, or as an anti-cancer agent, especially against leukemia, colon cancer, breast cancer, skin cancer and prostate cancer. In addition, due to their relatively high cell differentiation activity, these compounds provide a therapeutic agent for the treatment of various skin conditions including wrinkles, lack of adequate dermal hydration, i.e. dry skin, lack of adequate skin firmness, i.e. slack skin, and insufficient sebum secretion. Use of these compounds thus not only results in moisturizing of skin but also improves the barrier function of skin.

The compounds of the invention of formula I, and particularly formula Ia, are also useful in preventing or treating obesity, inhibiting adipocyte differentiation, inhibiting SCD-1 gene transcription, and/or reducing body fat in animal subjects. Therefore, in some embodiments, a method of preventing or treating obesity, inhibiting adipocyte differentiation, inhibiting SCD-1 gene transcription, and/or reducing body fat in an animal subject includes administering to the animal subject, an effective amount of one or more of the compounds or a pharmaceutical composition that includes one or more of the compounds of formula I. Administration of one or more of the compounds or the pharmaceutical compositions to the subject inhibits adipocyte differentiation, inhibits gene transcription, and/or reduces body fat in the animal subject.

One or more of the compounds may be present in a composition to treat the above-noted diseases and disorders in an amount from about 0.01 μg/gm to about 1000 μg/gm of the composition, preferably from about 0.1 μg/gm to about 500 μg/gm of the composition, and may be administered topically, transdermally, orally, rectally, nasally, sublingually or parenterally in dosages of from about 0.01μg/day to about 1000 μg/day, preferably from about 0.1 μg/day to about 500 μg/day.

### BRIEF DESCRIPTION OF THE DRAWINGS

In the drawings:

FIGS. 1-5 illustrate various biological activities of 2-methylene-20(21)-dehydro-19,24,25,26,27-pentanor-1α-hydroxyvitamin D₃, herein referred to as “MDBE 20,” as compared to the native hormone 1α,25-dihydroxyvitamin D₃, hereinafter “1,25(OH)₂D₃.”

FIG. 1 is a graph illustrating the relative activity of MDBE 20 and 1,25(OH)₂D₃ to compete for binding with [³H]-1,25-(OH)₂-D₃ to the full-length recombinant rat vitamin D receptor;

FIG. 2 is a graph illustrating the percent HL-60 cell differentiation as a function of the concentration of MDBE 20 and 1,25(OH)₂D₃;

FIG. 3 is a graph illustrating the in vitro transcription activity of 1,25(OH)₂D₃ as compared to MDBE 20;

FIG. 4 is a graph illustrating the bone calcium mobilization activity of 1,25(OH)₂D₃ as compared to MDBE 20; and

FIG. 5 is a graph illustrating the intestinal calcium transport activity of 1,25(OH)₂D₃ as compared to MDBE 20.

### DETAILED DESCRIPTION OF THE INVENTION

2-Methylene-20(21)-dehydro-19,24,25,26,27-pentanor-1α-hydroxyvitamin D₃ (referred to herein as “MDBE 20”) a 19-nor vitamin D analog which is characterized by the presence of a methylene substituent at carbon 2 (C-2), and a 1-methylene-propyl group substituted at carbon 17 (C-17), such that a double bond is located between carbon atom positions 20 and 21 in the side chain, was synthesized and tested. Such vitamin D analog seemed an interesting target because the relatively small methylene group at the C-2 position should not interfere with binding to the vitamin D receptor. Structurally, this 19-nor analog is characterized by the general formula Ia previously illustrated herein, and its pro-drug (in protected hydroxy form) is characterized by general formula I previously illustrated herein.

The preparation of 2-methylene-20(21)-dehydro-19-nor-vitamin D analogs having the structure I can be accomplished by a common general method, i.e. the condensation of a bicyclic Windaus-Grundmann type ketone II with the allylic phosphine oxide III to the corresponding 2-methylene-19-nor-vitamin D analog IV followed by deprotection at C-1 and C-3 in the latter compound (see Scheme I herein):

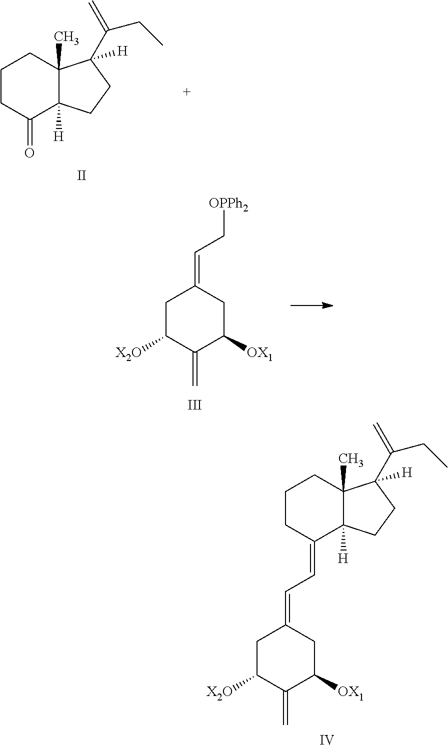

In the structures III and IV, groups X₁ and X₂ are hydroxy-protecting groups, preferably t-butyldimethylsilyl, it being also understood that any functionalities that might be sensitive, or that interfere with the condensation reaction, be suitably protected as is well-known in the art. The process shown above represents an application of the convergent synthesis concept, which has been applied effectively for the preparation of vitamin D compounds [e.g. Lythgoe et al., J. Chem. Soc. Perkin Trans. I, 590 (1978); Lythgoe, Chem. Soc. Rev. 9, 449 (1983); Toh et al., J. Org. Chem. 48, 1414 (1983); Baggiolini et al., J. Org. Chem. 51, 3098 (1986); Sardina et al., J. Org. Chem. 51, 1264 (1986); J. Org. Chem. 51, 1269 (1986); DeLuca et al., U.S. Pat. No. 5,086,191; DeLuca et al., U.S. Pat. No. 5,536,713].

The hydrindanone of the general structure II is not known. It can be prepared by the method shown in Scheme I herein (see the preparation of compound MDBE 20). For the preparation of the required hydrindanone of the structure II, a new synthetic route has been developed starting from the known [Fall et al., Tetrahedron Lett., 43, 1433 (2002); Granja et al., J. Org. Chem., 58, 124 (1993)] 22-aldehyde 1. A process involving transformation of the starting benzoyloxy aldehyde 1 into the desired C,D-ring synthon 7, and its subsequent coupling with the phosphine oxide 8, is summarized by the Scheme I. Thus, the aldehyde 1 was transformed into the mixture of isomeric E- and Z-oximes which on heating with acetic anhydride formed the expected nitrile 2. The nitrile was treated with LDA and the resulted carbanion alkylated by addition of ethyl bromide. The subsequent steps of the synthesis comprise the alkaline hydrolysis of 8β-benzoyloxy group in the obtained nitrile 3 producing the corresponding hydroxy nitrile 4. This process is desired in view of the following chemical transformation, i.e. DIBALH reduction of the C-20 cyano group leading to the hydroxy aldehyde 5. Direct DIBALH reduction of benzoyloxy nitrile 3 does not provide 5 in satisfactory yield whereas two-step procedure turns out to be significantly more efficient. Then, the aldehyde 5 was subjected to UV irradiation resulting in formaldehyde elimination. The obtained 8β-alcohol 6 was subsequently oxidized with tetrapropylammonium perruthenate to the hydrindanone 7. Wittig-Horner coupling of this Grundmann ketone with lithium phosphinoxy carbanion generated from the phosphine oxide 8 and phenyllithium gave the expected protected vitamin compound 9. This, after deprotection with tetrabutylammonium fluoride afforded 1α-hydroxy-2-methylene-20,21-dehydro-19,24,25,26,27-pentanorvitamin D₃ (10).

For the preparation of the required phosphine oxides of general structure III, a synthetic route has been developed starting from a methyl quinicate derivative which is easily obtained from commercial (1R,3R,4S,5R)-(−)-quinic acid as described by Perlman et al., Tetrahedron Lett. 32, 7663 (1991) and DeLuca et al., U.S. Pat. No. 5,086,191, and Sicinski et al, J. Med. Chem. 41, 4662 (1998).

The overall process of the synthesis of compounds I and Ia is illustrated and described more completely in U.S. Pat. No. 5,843,928 entitled “2-Alkylidene-19-Nor-Vitamin D Compounds” the specification of which is specifically incorporated herein by reference.

As used in the description and in the claims, the term “hydroxy-protecting group” signifies any group commonly used for the temporary protection of hydroxy functions, such as for example, alkoxycarbonyl, acyl, alkylsilyl or alkylarylsilyl groups (hereinafter referred to simply as “silyl” groups), and alkoxyalkyl groups. Alkoxycarbonyl protecting groups are alkyl-O—CO— groupings such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. The term “acyl” signifies an alkanoyl group of 1 to 6 carbons, in all of its isomeric forms, or a carboxyalkanoyl group of 1 to 6 carbons, such as an oxalyl, malonyl, succinyl, glutaryl group, or an aromatic acyl group such as benzoyl, or a halo, nitro or alkyl substituted benzoyl group. The word “alkyl” as used in the description or the claims, denotes a straight-chain or branched alkyl radical of 1 to 10 carbons, in all its isomeric forms. “Alkoxy” refers to any alkyl radical which is attached by oxygen, i.e. a group represented by “alkyl-O—. Alkoxyalkyl protecting groups are groupings such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals. The term “aryl” specifies a phenyl-, or an alkyl-, nitro- or halo-substituted phenyl group.

A “protected hydroxy” group is a hydroxy group derivatised or protected by any of the above groups commonly used for the temporary or permanent protection of hydroxy functions, e.g. the silyl, alkoxyalkyl, acyl or alkoxycarbonyl groups, as previously defined. The terms “hydroxyalkyl”, “deuteroalkyl” and “fluoroalkyl” refer to an alkyl radical substituted by one or more hydroxy, deuterium or fluoro groups respectively. An “alkylidene” refers to a radical having the general formula C_{K}H_{2K}—where K is an integer.

More specifically, reference should be made to the following illustrative example and description as well as to Scheme I herein for a detailed illustration of the preparation of compound MDBE 20.

In this example specific products identified by Arabic numerals (1, 2, 3) refer to the specific structures so identified in the Scheme I.

### EXAMPLE

Chemistry. Melting points (uncorrected) were determined on a Thomas-Hoover capillary melting-point apparatus. Ultraviolet (UV) absorption spectra were recorded with a Perkin-Elmer Lambda 3B UV-VIS spectrophotometer in ethanol. ¹H nuclear magnetic resonance (NMR) spectra were recorded at 400 and 500 MHz with a Bruker Instruments DMX-400 and DMX-500 Avance console spectrometers in deuteriochloroform. Chemical shifts (δ) are reported downfield from internal Me₄Si (δ0.00). Electron impact (EI) mass spectra were obtained with a Micromass AutoSpec (Beverly, Mass.) instrument. High-performance liquid chromatography (HPLC) was performed on a Waters Associates liquid chromatograph equipped with a Model 6000A solvent delivery system, a Model U6K Universal injector, and a Model 486 tunable absorbance detector. THF was freshly distilled before use from sodium benzophenone ketyl under argon.

### Preparation of 1α-hydroxy-20,21-dehydro-2-methylene-19,24,25,26,27-pentanor-vitamin D₃ (10)

Referring first to Scheme I the starting bicyclic aldehyde 1 was obtained according to the described procedure, Fall et al., Tetrahedron Lett., 43, 1433 (2002).

(a) Conversion of the Aldehyde 1 into 22-nitrile 2

Benzoic acid-(1R,3aR,4S,7aR)-1-((R)-cyano-methyl-methyl)-7a-methyl-octahydro-inden-4-yl ester (2). To a solution of a benzoyloxy aldehyde 1 (284 mg, 0.90 mmol) in anhydrous pyridine (5 mL) was added NH₂OH×HCl (210 mg) and the mixture was stirred at room temperature for 20 h. Then it was poured into water and extracted with ethyl acetate. The combined organic phases were separated, washed with saturated NaHCO₃ solution, water, and saturated CuSO₄ solution, dried (MgSO₄) and evaporated. The oily residue was purified by column chromatography on silica gel. Elution with hexane/ethyl acetate (9:1) gave pure, less polar E-oxime (167 mg) and more polar Z-oxime (105 mg, total yield 89%).
E-oxime: ¹H NMR (400 MHz, CDCl₃) δ 1.09 (3H, d, J=6.7 Hz, 18-H₃), 1.14 (3H, s, 21-H₃), 2.40 (1H, m, 20-H), 5.42 (1H, narr m, 8α-H), 7.27 (1H, d, J=8.0 Hz, 22-H), 7.45 (2H, t, J˜7 Hz, Ar—H), 7.56 (1H, t, J=7.4 Hz, Ar—H), 8.04 (2H, d, J=7.4 Hz, Ar—H).
Z-oxime: ¹H NMR (400 MHz, CDCl₃) δ 1.09 (3H, d, J=6.7 Hz, 18-H₃), 1.13 (3H, s, 21-H₃), 3.28 (1H, m, 20-H), 5.42 (1H, narr m, 8α-H), 6.25 (1H, d, J=8.1 Hz, 22-H), 7.45 (2H, t, J˜7 Hz, Ar—H), 7.56 (1H, t, J=7.3 Hz, Ar—H), 8.04 (2H, d, J=7.3 Hz, Ar—H).
The solution of the oximes (both isomers, 248 mg, 0.75 mmol) in acetic anhydride (8 mL) was refluxed for 1.5 h. The reaction mixture was cooled, poured carefully into saturated, water solution of NaHCO₃ and extracted with toluene. Extracts were combined, washed with water, dried (MgSO₄) and evaporated. The residue was applied on a silica Sep-Pak (5 g). Elution with hexane/ethyl acetate (95:5) gave pure semicrystalline nitrile 2 (212 mg, 91%). 2: [α]²⁴_{D}+81.5° (c 0.9 CHCl₃); ¹H NMR (400 MHz, CDCl₃) δ 1.124 (3H, s, 18-H₃), 1.373 (3H, d, J=7.1 Hz, 21-H₃), 1.90 (1H, br d, J=12.8 Hz, 9β-H), 2.68 (1H, pentet, J=7.0 Hz, 20-H), 5.43 (1H, narr m, 8α-H), 7.45 (2H, t, J=7.5 Hz, Ar—H), 7.57 (1H, t, J=7.5 Hz, Ar—H), 8.03 (2H, d, J=7.5 Hz, Ar—H); HRMS (ESI) exact mass calcd for C₁₃H₂₀ON (M⁺-C₆H₅CO) 206.1545, measured 206.1539.

(b) Alkylation of the Nitrile 2 with Ethyl Bromide

Benzoic acid-(1S,3aR,4S,7aR)-1-((S)-1-cyano-1-methyl-propyl)-7a-methyl-octahydro-inden-4-yl ester (3). n-BuLi (1.6 M in hexanes, 1.0 mL, 1.6 mmol) was added at 0° C. to the flask containing diisopropylamine (262 μL, 1.54 mmol) and THF (2 mL). The solution was stirred at 0° C. for 20 min., cooled to −78° C. and siphoned to the solution of 2 (430 mg, 1.31 mmol) in THF (1.5 mL). The resulted yellow mixture was stiffed for 30 min, then HMPA (600 μL) was added and stirring was continued for another 15 min. Then CH₃CH₂Br (310 μL, 4.08 mmol) was added, and the solution was stirred at −78° C. for 40 min. Saturated NH₄Cl was added and the mixture was extracted with ethyl acetate. The combined organic phases were washed with water, dried (MgSO₄) and evaporated. The residue was applied on a silica column. Elution with hexane/ethyl acetate (95:5) resulted in pure compound 3 (280 mg, 60%; 80% based on recovered substrate). Further elution with hexane/ethyl acetate (95:5) gave unreacted 2 (107 mg). 3: [α]²⁴_{D}+117.5° (c 0.2 CHCl₃); ¹H NMR (400 MHz, CDCl₃) δ 1.023 (3H, t, J=7.4 Hz, 23-H₃), 1.337 (3H, s, 18-H₃), 1.397 (3H, s, 21-H₃), 2.14 (1H, br d, J=12.9 Hz, 9β-H), 5.40 (1H, narr m, 8α-H), 7.45 (2H, t, J=7.4 Hz, Ar), 7.57 (1H, t, J=7.4 Hz, Ar), 8.05 (2H, d, J=7.4 Hz, Ar).

(c) Hydrolysis of the Benzoate 3

(S)-2-((1S,3aR,4S,7aR)-4-Hydroxy-7a-methyl-octahydro-inden-1-yl)-2-methyl-butyronitrile (4). A solution of the benzoyloxy nitrile 3 (270 mg, 0.76 mmol) in 10% KOH in MeOH (12 mL) was heated at 50° C. for 18 h, poured into water and extracted with ethyl acetate. Organic phase was washed with NaHCO₃, water, dried (MgSO₄) and evaporated. The oily residue was purified on a silica Sep-Pak (2 g). Elution with hexane/ethyl acetate (8:2) gave pure hydroxy nitrile 4 (179 mg, 99%). 4: [α]²⁴_{D}+26.5° (c 0.33 CHCl₃); ¹H NMR (400 MHz, CDCl₃) δ 1.004 (3H, t, J=7.3 Hz, 23-H₃), 1.349 (3H, s, 21-H₃), 1.240 (s, 18-H₃), 4.10 (1H, narr m, 8α-H).

(d) Reduction of the Nitrile 4 with DIBALH

(S)-2-((1S,3aR,4S,7aR)-4-Hydroxy-7a-methyl-octahydro-inden-1-yl)-2-methyl-butyraldehyde (5). To the solution of nitrile 4 (172 mg, 0.773 mmol) in anhydrous methylene chloride (3.3 mL) was slowly added solution of diisobutylaluminum hydride (1.5 M in toluene; 1.66 mL, 2.3 mmol) at −78° C. The solution was stirred for 1 h, and then it was allowed to warm up to −30° C. during 1 h 30 and was cooled to −78° C. again. The reaction was quenched by addition of brine containing 5% HCl and it was extracted with ethyl acetate. The combined organic layers were washed with NaHCO₃ and brine, dried (MgSO₄) and evaporated. The remaining residue was purified on a silica Sep-Pak (2 g). Elution with hexane/ethyl acetate (8:2) gave the pure hydroxy aldehyde 38 (112 mg, 65%). 5: [α]²⁴_{D}+5° (c (0.25 CHCl₃); ¹H NMR (400 MHz, CDCl₃) δ 0.781 (3H, t, J=7.3 Hz, 23-H₃), 0.965 (3H, s, 21-H₃), 1.105 (3H, s, 18-H₃), 2.02 (1H, br d, J=14.2 Hz, 9β-H), 4.09 (1H, narr m, 8α-H), 9.72 (1H, s, CHO); HRMS (ESI) exact mass calcd for C₁₄H₂₆O (M⁺+Na) 261.1831, measured 261.1847.

(e) Irradiation of the Aldehyde 5

(1R,3aR,4S,7aR)-1-(1-Methylene-propyl)-7a-methyl-octahydro-inden-4-ol (6). A solution of aldehyde 5 (24 mg, 0.10 mmol) in hexane (about 350 mL), in an apparatus consisting of a Pyrex vessel, was cooled to 0° C. and degassed with argon. It was irradiated for 150 min through a water-cooled (0° C.) quartz inner well with a 350 W Hanau S 81 mercury arc lamp. The solution was allowed to warm to room temperature, concentrated under vacuum and applied on silica column. Elution with hexane/ethyl acetate (97:3) resulted in mixture of decarbonylation products (3.3 mg) and the olefin 6 (2.0 mg). Final purification of product 6 was achieved by HPLC (10 mm×25 cm, Zorbax-Sil column, 4 mL/min), using hexane/ethyl acetate (88:12). Alcohol 6 was collected at Rᵥ=30 mL. 6: [α]²⁴D+10° (c, 0.17, CHCl₃); ¹H NMR (400 MHz, CDCl₃) δ 0.864 (3H, s, 18-H₃), 1.02 (3H, t, J=7.4 Hz, 23-H₃), 4.10 (1H, narr m, 8β-H), 4.77 and 4.92 (each 1H, each s, H₂C═C).

(f) Oxidation of Alcohol 6

(1R,3aR,7aR)-1-(1-Methylene-propyl)-7a-methyl-octahydro-inden-4-one (7). The solution of NMO (3.9 mg) and molecular sieves 4 Å (22 mg) in methylene chloride (0.2 mL) was stirred at room temperature for 15 min., then the solution of 6 (3.2 mg, 15.4 μmol) in methylene chloride (0.15 mL) was added followed by TPAP (0.5 mg). The resulted dark mixture was stirred for 20 min, diluted with methylene chloride and filtered through a silica Sep-Pak (2 g). Elution with methylene chloride gave ketone 7 (2.9 mg, 91%). 7: ¹H NMR (500 MHz, CDCl₃) δ 0.553 (3H, s, 18-H₃), 1.04 (3H, t, J=7.3 Hz, 23-H₃), 2.55 (1H, dd, J=10.1, 7.9 Hz, 14α-H), 4.83 and 4.92 (each 1H, each s, H₂C═C).

(f) Wittig-Horner Coupling of the Ketone 7 with the Phosphine Oxide 8

1α-[(tert-Butyldimethylsilyl)oxy]-20,21-dehydro-2-methylene-19,24,25,26,27-pentanorvitamin D₃ tert-Butyldimethylsilyl Ether (9). To a solution of phosphine oxide 8 (32 mg, 57 μmol) in anhydrous THF (0.60 mL) at −78° C. was slowly added phenyllithium (1.8 M in butyl ether, 32 μL, 57 μmol) under argon with stirring. The solution turned deep orange. The mixture was stirred at −78° C. for 20 min and a precooled (−78° C.) solution of the ketone 7 (2.7 mg, 13 μmol) in anhydrous THF (0.10 mL) was slowly added. The mixture was stirred under argon at −78° C. for 1 h and during 1 h 30 min warmed to −20° C. Ethyl acetate and water were added, and the organic phase was washed with brine, dried (MgSO₄), and evaporated. The residue was dissolved in hexane, applied on a silica Sep-Pak, and washed with hexane/ethyl acetate (99:1) to give silylated vitamin 9 (1.5 mg, 20%). The column was then washed with hexane/ethyl acetate to recover the phosphine oxide 8 (22 mg). 9: ¹H NMR (400 MHz, CDCl₃) δ 0.029, 0.048, 0.070 and 0.079 (each 3H, each s, 4×SiCH₃), 0.448 (3H, s, 18-H₃), 0.866 and 0.896 (each 9H, each s, 2×Si-t-Bu), 1.03 (3H, t, J=7.3 Hz, 23-H₃), 2.51 (1H, dd, J=13.2, 6.0 Hz, 4β-H), 2.46 (1H, dd, J=12.8, 4.3 Hz, 10β-H), 2.35 (1H, dd, J=13.2, 3.1 Hz, 4α-H), 2.26 (1H, t, J=9.6 Hz, 10α-H), 2.84 (1H, br d, J˜12 Hz, 9β-H), 4.43 (2H, m, 1β- and 3α-H), 4.81, 4.87, 4.92 and 4.97 (each 1H, each s, 2×H₂C═C), 5.86 and 6.22 (1H and 1H, each d, J=11.2 Hz, 7- and 6-H).

(g) Hydrolysis of the Silyl Protecting Groups in the 19-norvitamin D Derivative 9

1α-Hydroxy-20,21-dehydro-2-methylene-19,24,25,26,27-pentanorvitamin D₃ (10). To a solution of the protected vitamin 9 (1.4 mg, 2.45 μmol) in anhydrous THF (1.5 mL) was added tetrabutylammonium fluoride (1.0 M in THF, 70 μL, 70 μmol) and triethylamine (10 μL). The mixture was stirred under argon at room temperature for 18 h, poured into brine and extracted with ethyl acetate and diethyl ether. Organic extracts were washed with brine, dried (MgSO₄), and evaporated. The residue was purified by HPLC (9.4 mm×25 cm Zorbax-Sil column, 4 mL/min) using hexane/2-propanol (9:1) solvent system. Pure 19-norvitamin 10 (0.76 mg, 85%) was collected at Rᵥ 25.7 mL. In reversed-phase HPLC (9.4 mm×25 cm Eclipse XDB-C18 column, 3 mL/min) using methanol/water (95:5) solvent system the vitamin 10 was collected at Rᵥ 22.0 mL. 10 (MDB20): UV (in EtOH) λₘₐₓ 245.0, 252.5, 262.0 nm; ¹H NMR (400 MHz, CDCl₃) δ 0.453 (3H, s, 18-H₃), 1.04 (3H, t, J=7.3 Hz, 23-H₃), 2.27 (2H, m), 2.34 (1H, dd, J=13.2, 6.0 Hz, 4β-H), 2.57 (1H, dd, J=13.2, 4.0 Hz, 4α-H), 2.80 (1H, br d, J˜12.5 Hz, 9β-H), 4.48 (2H, m, 1β- and 3α-H), 4.80, 4.87, 5.09 and 5.11 (each 1H, each s, H₂C═C), 5.90 and 6.36 (1H and 1H, each d, J=11.2 Hz, 7- and 6-H).

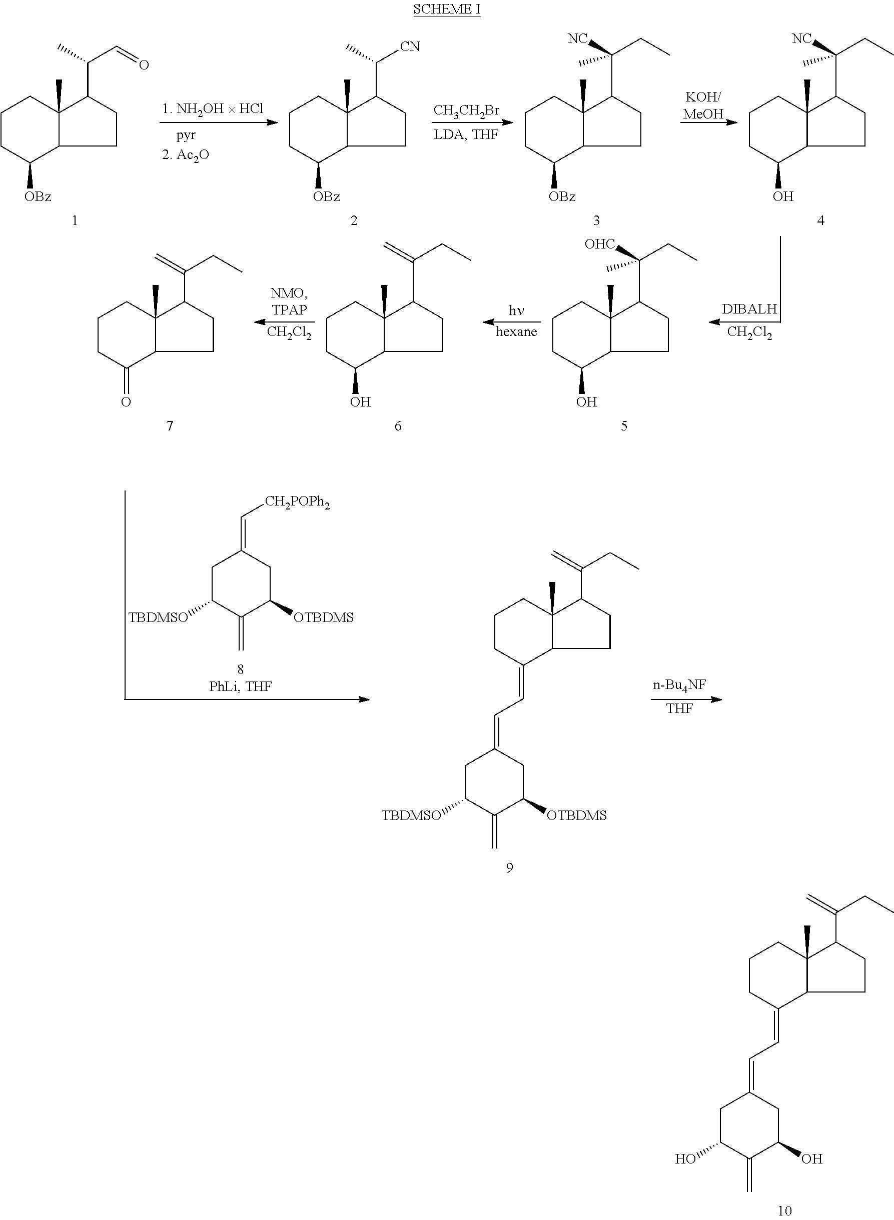

### Biological Activity of 2-Methylene-20(21)-Dehydro-19,24,25,26,27-Pentanor-1α-Hydroxyvitamin D₃ (MDBE 20)

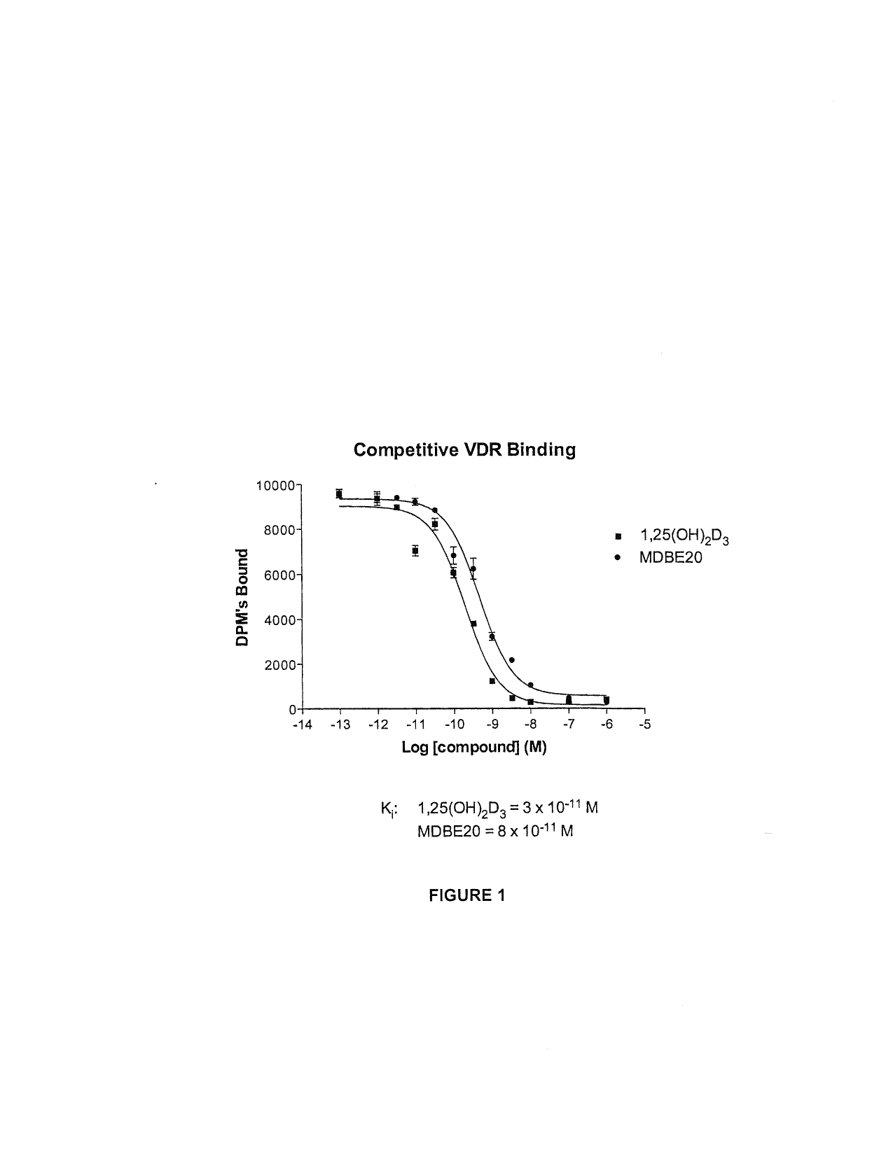

The introduction of a methylene group to the 2-position, and a 1-methylene-propyl group substituted at carbon 17, such that a double bond is between carbon atoms 20 and 21 in the side chain, had little effect on binding of MDBE 20 to the full length recombinant rat vitamin D receptor, as compared to 1α,25-dihydroxyvitamin D₃. The compound MDBE 20 bound with almost the same affinity to the nuclear vitamin D receptor as compared to the standard 1,25-(OH)₂D₃ (FIG. 1). It might be expected from these results that compound MDBE 20 would have equivalent biological activity. Surprisingly, however, compound MDBE 20 is a highly selective analog with unique biological activity.

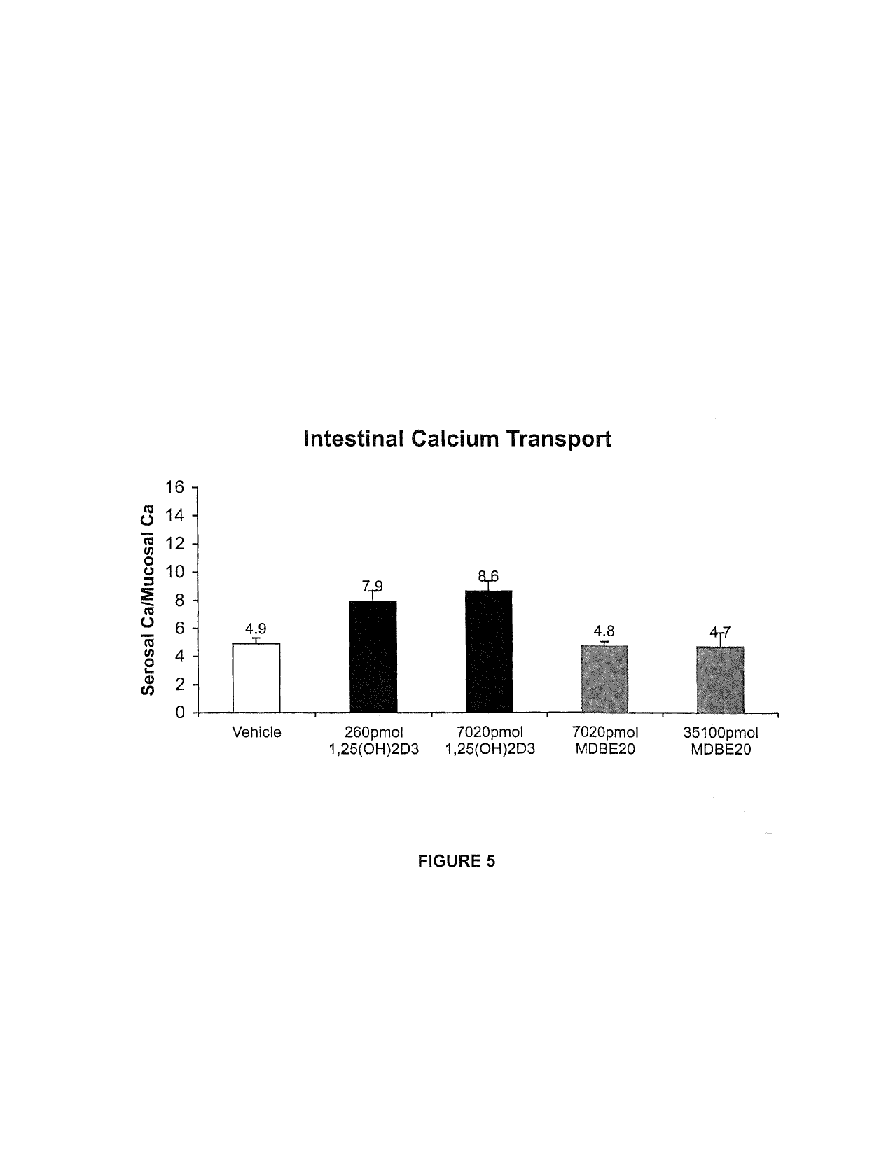

FIG. 5 shows that MDBE 20 has little, if any, ability to increase intestinal calcium transport activity in vivo. It has substantially no activity as compared to that of 1,25-dihydroxyvitamin D₃ (1,25(OH)₂D₃), the natural hormone, in stimulating intestinal calcium transport, even at the 35,100 pmol dose.

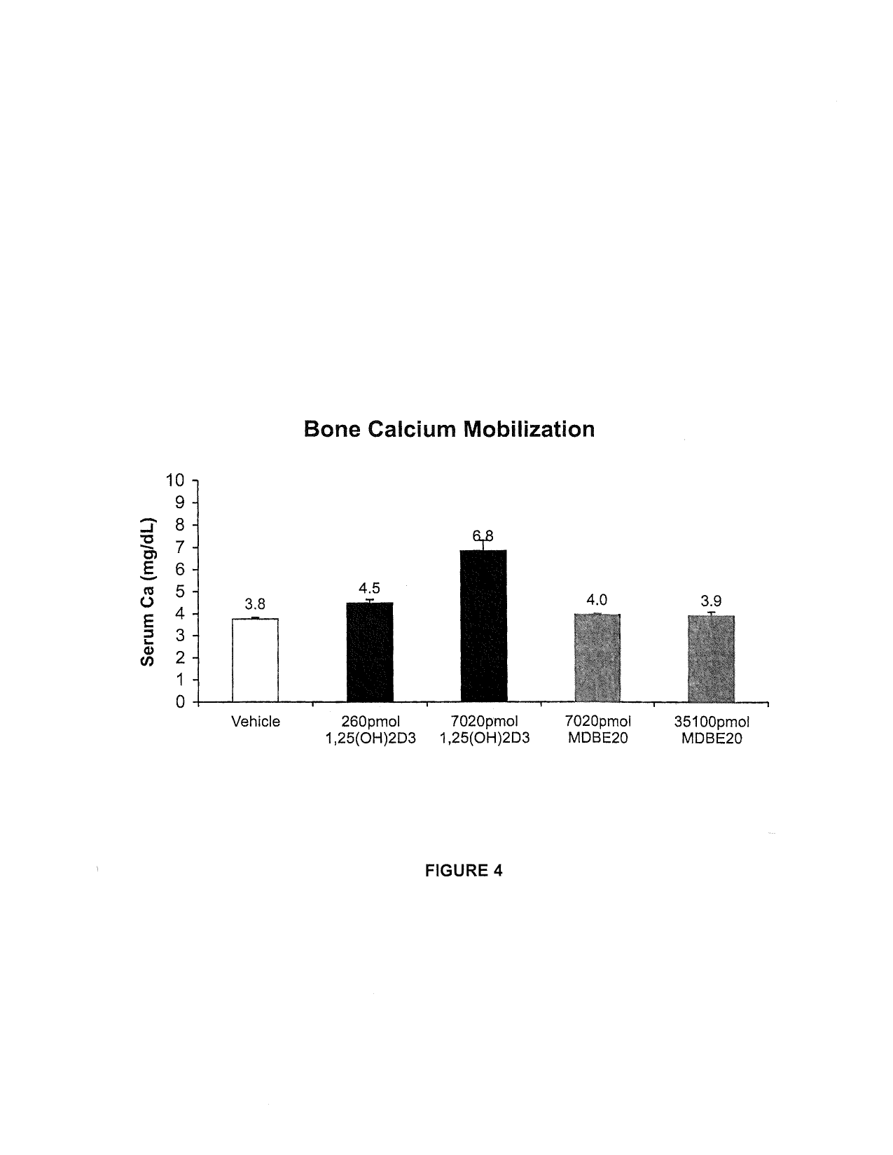

FIG. 4 demonstrates that MDBE 20 has little, if any, bone calcium mobilization activity, as compared to 1,25(OH)₂D₃. MDBE 20 has no measurable bone calcium mobilization activity, at the dose levels tested, as compared to 1,25(OH)₂D₃.

FIGS. 4 and 5 thus illustrate that MDBE 20 may be characterized as having no significant intestinal calcium transport activity, and no significant bone calcium mobilization activity.

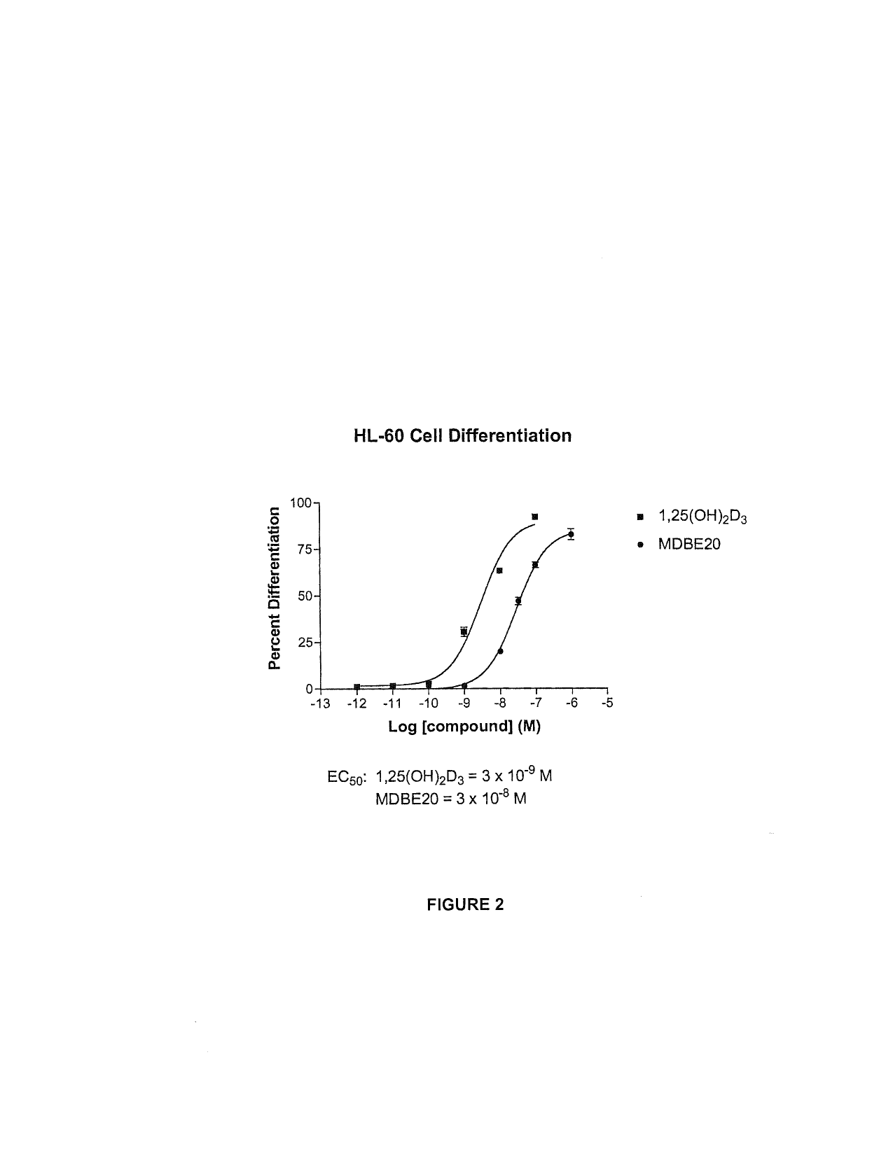

FIG. 2 illustrates that MDBE 20 is only about 10 times less potent (about one log less potent) than 1,25(OH)₂D₃ on HL-60 cell differentiation, i.e. causing the differentiation of HL-60 cells into monocytes, making it an excellent candidate for the treatment of psoriasis and cancer, especially against leukemia, colon cancer, breast cancer, skin cancer and prostate cancer. In addition, due to its relatively high cell differentiation activity, this compound provides a therapeutic agent for the treatment of various skin conditions including wrinkles, lack of adequate dermal hydration, i.e. dry skin, lack of adequate skin firmness, i.e. slack skin, and insufficient sebum secretion. Use of this compound thus not only results in moisturizing of skin but also improves the barrier function of skin.

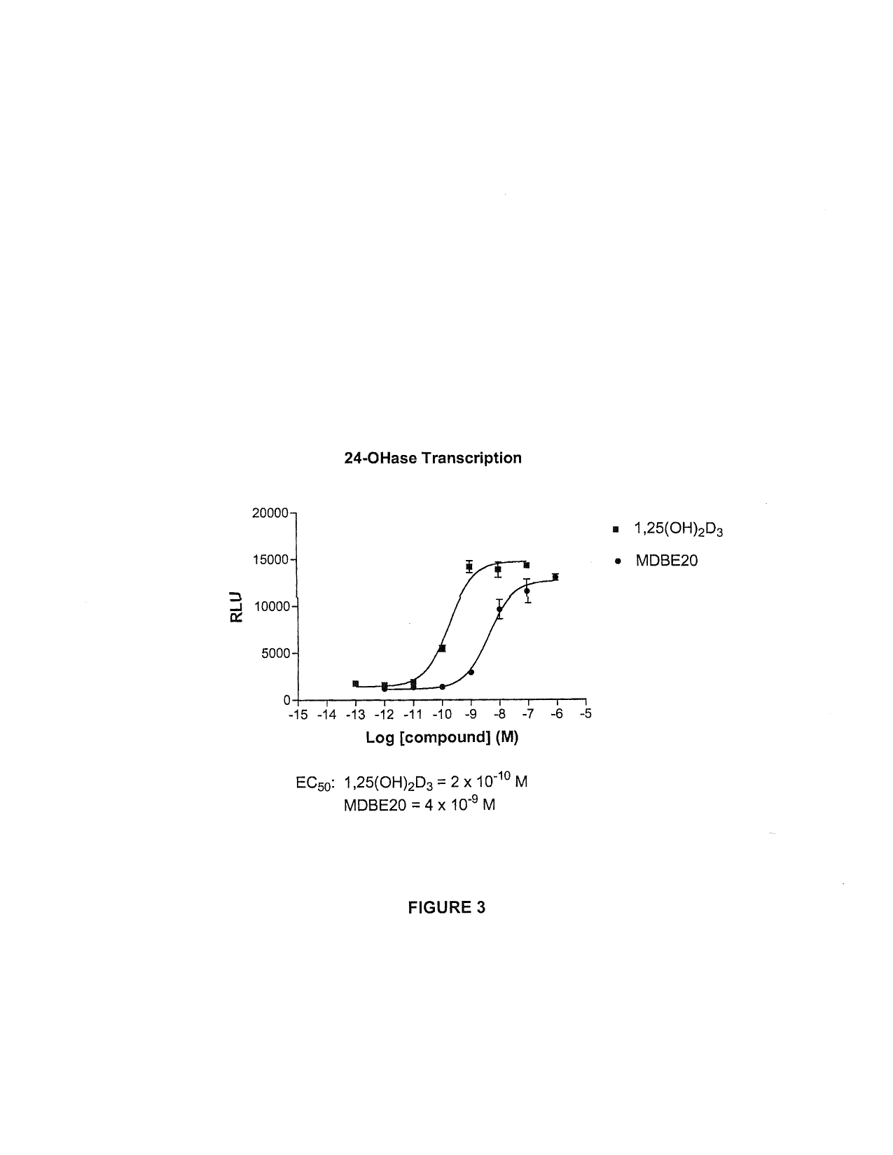

FIG. 3 illustrates that in bone cells the compound MDBE 20 is one log, i.e. 10 times, less potent than 1,25(OH)₂D₃ in increasing transcription of the 24-hydroxylase gene. This result, together with the cell differentiation activity of FIG. 2, suggests that MDBE 20 will be very effective in psoriasis because it has direct cellular activity in causing cell differentiation, gene transcription, and in suppressing cell growth. These data also indicate that MDBE 20 may have significant activity as an anti-cancer agent, especially against leukemia, colon cancer, breast cancer, skin cancer and prostate cancer.

The strong activity of MDBE 20 on HL-60 differentiation suggests it will be active in suppressing growth of parathyroid glands and in the suppression of the preproparathyroid gene, indicating these compounds may be useful as a therapy for suppression of secondary hyperparathyroidism caused by chronic renal failure as well as a treatment for renal osteodystrophy.

### Experimental Methods

Vitamin D Receptor Binding

Test Material

Protein Source

Full-length recombinant rat receptor was expressed in E. coli BL21 (DE3) Codon Plus RIL cells and purified to homogeneity using two different column chromatography systems. The first system was a nickel affinity resin that utilizes the C-terminal histidine tag on this protein. The protein that was eluted from this resin was further purified using ion exchange chromatography (S-Sepharose Fast Flow). Aliquots of the purified protein were quick frozen in liquid nitrogen and stored at −80° C. until use. For use in binding assays, the protein was diluted in TEDK₅₀ (50 mM Tris, 1.5 mM EDTA, pH7.4, 5 mM DTT, 150 mM KCl) with 0.1% Chaps detergent. The receptor protein and ligand concentration were optimized such that no more than 20% of the added radiolabeled ligand was bound to the receptor.

Study Drugs

Unlabeled ligands were dissolved in ethanol and the concentrations determined using UV spectrophotometry (1,25(OH)₂D₃: molar extinction coefficient=18,200 and λₘₐₓ=265 nm; Analogs: molar extinction coefficient=42,000 and λₘₐₓ=252 nm). Radiolabeled ligand (³H-1,25(OH)₂D₃, ˜159 Ci/mmole) was added in ethanol at a final concentration of 1 nM.

Assay Conditions

Radiolabeled and unlabeled ligands were added to 100 mcl of the diluted protein at a final ethanol concentration of ≦10%, mixed and incubated overnight on ice to reach binding equilibrium. The following day, 100 mcl of hydroxylapatite slurry (50%) was added to each tube and mixed at 10-minute intervals for 30 minutes. The hydroxylapaptite was collected by centrifugation and then washed three times with Tris-EDTA buffer (50 mM Tris, 1.5 mM EDTA, pH 7.4) containing 0.5% Titron X-100. After the final wash, the pellets were transferred to scintillation vials containing 4 ml of Biosafe II scintillation cocktail, mixed and placed in a scintillation counter. Total binding was determined from the tubes containing only radiolabeled ligand.

HL-60 Differentiation

Test Material

Study Drugs

The study drugs were dissolved in ethanol and the concentrations determined using UV spectrophotometry. Serial dilutions were prepared so that a range of drug concentrations could be tested without changing the final concentration of ethanol (≦0.2%) present in the cell cultures.

Cells

Human promyelocytic leukemia (HL60) cells were grown in RPMI-1640 medium containing 10% fetal bovine serum. The cells were incubated at 37° C. in the presence of 5% CO₂.

Assay Conditions

HL60 cells were plated at 1.2×10⁵ cells/ml. Eighteen hours after plating, cells in duplicate were treated with drug. Four days later, the cells were harvested and a nitro blue tetrazolium reduction assay was performed (Collins et al., 1979; J. Exp. Med. 149:969-974). The percentage of differentiated cells was determined by counting a total of 200 cells and recording the number that contained intracellular black-blue formazan deposits. Verification of differentiation to monocytic cells was determined by measuring phagocytic activity (data not shown).

In vitro Transcription Assay

Transcription activity was measured in ROS 17/2.8 (bone) cells that were stably transfected with a 24-hydroxylase (24Ohase) gene promoter upstream of a luciferase reporter gene (Arbour et al., 1998). Cells were given a range of doses. Sixteen hours after dosing the cells were harvested and luciferase activities were measured using a luminometer.

RLU=relative luciferase units.

Intestinal Calcium Transport and Bone Calcium Mobilization

Male, weanling Sprague-Dawley rats were placed on Diet 11 (0.47% Ca) diet+AEK oil for one week followed by Diet 11 (0.02% Ca)+AEK oil for 3 weeks. The rats were then switched to a diet containing 0.47% Ca for one week followed by two weeks on a diet containing 0.02% Ca. Dose administration began during the last week on 0.02% calcium diet. Four consecutive ip doses were given approximately 24 hours apart. Twenty-four hours after the last dose, blood was collected from the severed neck and the concentration of serum calcium determined as a measure of bone calcium mobilization. The first 10 cm of the intestine was also collected for intestinal calcium transport analysis using the everted gut sac method.

### Interpretation of Data

Summary of Biological Findings. The compound MDBE 20 binds the VDR with almost the same affinity as the native hormone, and displays only about 1 log (10 times) less cell differentiation activity and only about 1 log (10 times) less in vitro gene transcription activity compared to 1,25(OH)₂D₃. In vivo this compound exhibits significantly less bone calcium mobilization activity and significantly less intestinal calcium transport activity compared to the native hormone making this compound a potentially valuable agent for the treatment of such diseases as cancer, renal osteodystrophy, secondary hyperparathyroidism, autoimmune diseases, skin conditions, and psoriasis. While this compound is almost as potent compared to 1,25(OH)₂D₃ in vitro, it shows no activity in vivo on bone calcium mobilization and in the intestine compared to the native hormone. MDBE 20 remains a potentially valuable compound for therapeutic development as it has little intestinal calcium transport activity as well as little potency in mobilizing calcium from bone, but relatively high potency in cell differentiation potentially resulting in a compound with a larger safety window than has previously been generated. MDBE 20 might not only be useful in the treatment of the above listed diseases, but also in the prevention of the above listed diseases.

VDR binding, HL60 cell differentiation, and transcription activity. MDBE 20 (Kᵢ=8×10⁻¹¹M) is almost as active (about one-half log less active) as the natural hormone 1α,25-dihydroxyvitamin D₃ (Kᵢ=3×10⁻¹¹M) in its ability to compete with [³H]-1,25(OH)₂D₃ for binding to the full-length recombinant rat vitamin D receptor (FIG. 1). MDBE 20 displays only about 10 times (1 log) less activity (EC₅₀=3×10⁻⁸M) in its ability (efficacy or potency) to promote HL-60 cell differentiation as compared to 1α,25-dihydroxyvitamin D₃ (EC₅₀=3×10⁻⁹M) (See FIG. 2). Also, compound MDBE 20 (EC₅₀=4×10⁻⁹M) has less than 10 times the transcriptional activity in bone cells than 1α,25-dihydroxyvitamin D₃ (EC₅₀=2×10⁻¹⁰M) (See FIG. 3). These results suggest that MDBE 20 will be very effective in psoriasis because it has direct cellular activity in causing cell differentiation, gene transcription, and in suppressing cell growth. These data also indicate that MDBE 20 will have significant activity as an anti-cancer agent, especially against leukemia, colon cancer, breast cancer, skin cancer and prostate cancer, as well as against skin conditions such as dry skin (lack of dermal hydration), undue skin slackness (insufficient skin firmness), insufficient sebum secretion and wrinkles. It would also be expected to be very active in suppressing secondary hyperparathyroidism.

Calcium mobilization from bone and intestinal calcium absorption in vitamin D-deficient animals. Using vitamin D-deficient rats on a low calcium diet (0.02%), the activities of MDBE 20 and 1,25(OH)₂D₃ in intestine and bone were tested. As expected, the native hormone (1,25(OH)₂D₃) increased serum calcium levels at the 260 pmol/day dosage (FIG. 4). The study reported in FIG. 4 shows that MDBE 20 has relatively low or little activity in mobilizing calcium from bone. Administration MDBE 20 at 7020 pmol/day for 4 consecutive days did not result in mobilization of bone calcium, and even when the amount of MDBE 20 was increased to 35100 pmol/day no substantial effect was seen.

Intestinal calcium transport was evaluated in the same groups of animals using the everted gut sac method (FIG. 5). These results show that the compound MDBE 20 does not promote intestinal calcium transport when administered at 7020 or 35100 pmol/day, and its activity is significantly less than 1,25(OH)₂D₃ which provides a significant increase at the 260 pmol/day dose. Thus, it may be concluded that MDBE 20 has no intestinal calcium transport activity at the recommended lower doses as compared to 1,25-(OH)₂D₃.

These results illustrate that MDBE 20 is an excellent candidate for numerous human therapies as described herein, and that it may be particularly useful in a number of circumstances such as suppression of secondary hyperparathyroidism of renal osteodystrophy, autoimmune diseases, cancer, renal osteodystrophy, numerous types of skin conditions, and psoriasis. MDBE 20 is an excellent candidate for treating psoriasis because: (1) it has significant VDR binding, transcription activity and cellular differentiation activity; (2) it has little hypercalcemic liability at relatively low doses, unlike 1,25(OH)₂D₃; and (3) it is easily synthesized. Since MDBE 20 has significant binding activity to the vitamin D receptor, but has little ability to raise blood serum calcium, it may also be particularly useful for the treatment of secondary hyperparathyroidism of renal osteodystrophy, as well as treatment of renal osteodystrophy per se.

These data also indicate that the compound MDBE 20 of the invention may be especially suited for treatment and prophylaxis of human disorders which are characterized by an imbalance in the immune system, e.g. in autoimmune diseases, including multiple sclerosis, lupus, diabetes mellitus, host versus graft rejection, and rejection of organ transplants; and additionally for the treatment of inflammatory diseases, such as rheumatoid arthritis, asthma, and inflammatory bowel diseases such as celiac disease, ulcerative colitis and Crohn's disease.

The compounds of the invention of formula I, and particularly formula Ia, are also useful in preventing or treating obesity, inhibiting adipocyte differentiation, inhibiting SCD-1 gene transcription, and/or reducing body fat in animal subjects. Therefore, in some embodiments, a method of preventing or treating obesity, inhibiting adipocyte differentiation, inhibiting SCD-1 gene transcription, and/or reducing body fat in an animal subject includes administering to the animal subject, an effective amount of one or more of the compounds or a pharmaceutical composition that includes one or more of the compounds of formula I. Administration of the compound MDBE 20 or the pharmaceutical compositions to the subject inhibits adipocyte differentiation, inhibits gene transcription, and/or reduces body fat in the animal subject. The animal may be a human, a domestic animal such as a dog or a cat, or an agricultural animal, especially those that provide meat for human consumption, such as fowl like chickens, turkeys, pheasant or quail, as well as bovine, ovine, caprine, or porcine animals.

For prevention and/or treatment purposes, the compounds of this invention defined by formula I, particulary MDBE 20, may be formulated for pharmaceutical applications as a solution in innocuous solvents, or as an emulsion, suspension or dispersion in suitable solvents or carriers, or as pills, tablets or capsules, together with solid carriers, according to conventional methods known in the art. Any such formulations may also contain other pharmaceutically-acceptable and non-toxic excipients such as stabilizers, anti-oxidants, binders, coloring agents or emulsifying or taste-modifying agents.

The compounds of formula I and particularly MDBE 20, may be administered orally, topically, parenterally, rectally, nasally, sublingually or transdermally. The compound is advantageously administered by injection or by intravenous infusion or suitable sterile solutions, or in the form of liquid or solid doses via the alimentary canal, or in the form of creams, ointments, patches, or similar vehicles suitable for transdermal applications. A dose of from 0.01 μg to 1000 μg per day of the compounds I, particularly MDBE 20, preferably from about 0.1 μg to about 500 μg per day, is appropriate for prevention and/or treatment purposes, such dose being adjusted according to the disease to be treated, its severity and the response of the subject as is well understood in the art. Since the compound exhibits specificity of action, each may be suitably administered alone, or together with graded doses of another active vitamin D compound—e.g. 1α-hydroxyvitamin D₂ or D₃, or 1α,25-dihydroxyvitamin D₃—in situations where different degrees of bone mineral mobilization and calcium transport stimulation is found to be advantageous.

Compositions for use in the above-mentioned treatments comprise an effective amount of the compounds I, particularly MDBE 20, as defined by the above formula I and Ia as the active ingredient, and a suitable carrier. An effective amount of such compound for use in accordance with this invention is from about 0.01 μg to about 1000 μg per gm of composition, preferably from about 0.1 μg to about 500 μg per gram of composition, and may be administered topically, transdermally, orally, rectally, nasally, sublingually, or parenterally in dosages of from about 0.01μg/day to about 1000 μg/day, and preferably from about 0.1 μg/day to about 500 μg/day.

The compounds I, particularly MDBE 20, may be formulated as creams, lotions, ointments, topical patches, pills, capsules or tablets, suppositories, aerosols, or in liquid form as solutions, emulsions, dispersions, or suspensions in pharmaceutically innocuous and acceptable solvent or oils, and such preparations may contain in addition other pharmaceutically innocuous or beneficial components, such as stabilizers, antioxidants, emulsifiers, coloring agents, binders or taste-modifying agents.

The compounds I, particularly MDBE 20, may be advantageously administered in amounts sufficient to effect the differentiation of promyelocytes to normal macrophages. Dosages as described above are suitable, it being understood that the amounts given are to be adjusted in accordance with the severity of the disease, and the condition and response of the subject as is well understood in the art.

The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof.

Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion.

Formulations for rectal administration may be in the form of a suppository incorporating the active ingredient and carrier such as cocoa butter, or in the form of an enema.

Formulations suitable for parenteral administration conveniently comprise a sterile oily or aqueous preparation of the active ingredient which is preferably isotonic with the blood of the recipient.

Formulations suitable for topical administration include liquid or semi-liquid preparations such as liniments, lotions, applicants, oil-in-water or water-in-oil emulsions such as creams, ointments or pastes; or solutions or suspensions such as drops; or as sprays.

For nasal administration, inhalation of powder, self-propelling or spray formulations, dispensed with a spray can, a nebulizer or an atomizer can be used. The formulations, when dispensed, preferably have a particle size in the range of 10 to 100μ.

The formulations may conveniently be presented in dosage unit form and may be prepared by any of the methods well known in the art of pharmacy. By the term “dosage unit” is meant a unitary, i.e. a single dose which is capable of being administered to a patient as a physically and chemically stable unit dose comprising either the active ingredient as such or a mixture of it with solid or liquid pharmaceutical diluents or carriers.

I claim:

1. A compound having the formula: 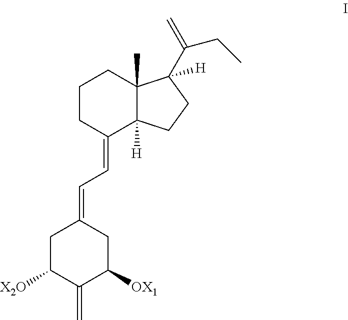
where X₁ and X₂, which may be the same or different, are each selected from hydrogen or a hydroxy-protecting group.

2. The compound of claim 1 wherein X₂ is hydrogen.

3. The compound of claim 1 wherein X₁ is hydrogen.

4. The compound of claim 1 wherein X₁ and X₂ are both t-butyldimethylsilyl.

5. A pharmaceutical composition containing an effective amount of at least one compound as claimed in claim 1 together with a pharmaceutically acceptable excipient.

6. The pharmaceutical composition of claim 5 wherein said effective amount comprises from about 0.01 μg to about 1000 μg per gram of composition.

7. The pharmaceutical composition of claim 5 wherein said effective amount comprises from about 0.1 μg to about 500 μg per gram of composition.

8. A compound having the formula: 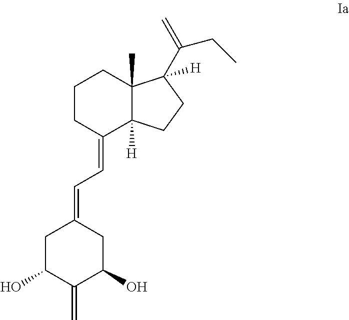 [SMILES: C=C1[C@H](O)CC(=C/C=C2\CCC[C@]3(C)[C@@H](C(=C)CC)CC[C@@H]23)C[C@H]1O]
and is named 2-methylene-20(21)-dehydro-19,24,25,26,27-pentanor-1α-hydroxyvitamin D₃.

9. A pharmaceutical composition containing an effective amount of 2-methylene-20(21)-dehydro-19,24,25,26,27-pentanor-1α-hydroxyvitamin D₃ together with a pharmaceutically acceptable excipient.

10. The pharmaceutical composition of claim 9 wherein said effective amount comprises from about 0.01 μg to about 1000 μg per gram of composition.

11. The pharmaceutical composition of claim 9 wherein said effective amount comprises from about 0.1 μg to about 500 μg per gram of composition.

12. A method of treating psoriasis comprising administering to a subject with psoriasis an effective amount of a compound having the formula: 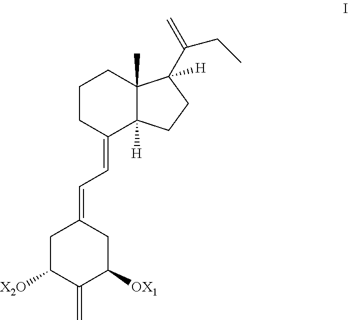
where X₁ and X₂ which may be the same or different, are each selected from hydrogen or a hydroxy-protecting group.

13. The method of claim 12 wherein the compound is administered orally.

14. The method of claim 12 wherein the compound is administered parenterally.

15. The method of claim 12 wherein the compound is administered transdermally.

16. The method of claim 12 wherein the compound is administered topically.

17. The method of claim 12 wherein the compound is administered rectally.

18. The method of claim 12 wherein the compound is administered nasally.

19. The method of claim 12 wherein the compound is administered sublingually.

20. The method of claim 12 wherein the compound is administered in a dosage of from about 0.01 μg/day to about 1000 μg/day.

21. The method of claim 12 wherein the compound has the formula: 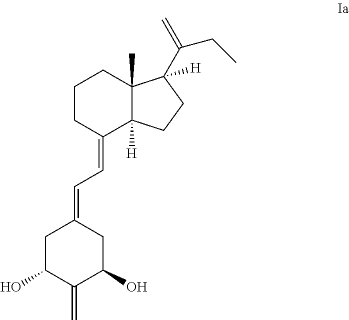 [SMILES: C=C1[C@H](O)CC(=C/C=C2\CCC[C@]3(C)[C@@H](C(=C)CC)CC[C@@H]23)C[C@H]1O]
and is named 2-methylene-20(21)-dehydro-19,24,25,26,27-pentanor-1α-hydroxyvitamin D₃

22. A method of treating a disease selected from the group consisting of leukemia, colon cancer, breast cancer, skin cancer or prostate cancer comprising administering to a subject with said disease an effective amount of a compound having the formula: 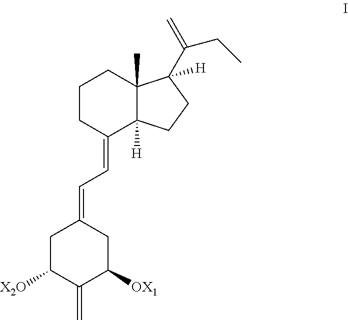
where X₁ and X₂, which may be the same or different, are each selected from hydrogen or a hydroxy-protecting group.

23. The method of claim 22 wherein the compound is administered orally.

24. The method of claim 22 wherein the compound is administered parenterally.

25. The method of claim 22 wherein the compound is administered transdermally.

26. The method of claim 22 wherein the compound is administered rectally.

27. The method of claim 22 wherein the compound is administered nasally.

28. The method of claim 22 wherein the compound is administered sublingually.

29. The method of claim 22 wherein the compound is administered in a dosage of from about 0.01 μg/day to about 1000 μg/day.

30. The method of claim 22 wherein the compound has the formula: 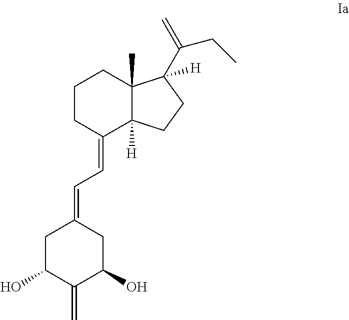 [SMILES: C=C1[C@H](O)CC(=C/C=C2\CCC[C@]3(C)[C@@H](C(=C)CC)CC[C@@H]23)C[C@H]1O]
and is named 2-methylene-20(21)-dehydro-19,24,25,26,27-pentanor-1α-hydroxyvitamin D₃.

31. A method of treating an autoimmune disease selected from the group consisting of multiple sclerosis, lupus, diabetes mellitus, host versus graft rejection, and rejection of organ transplants, comprising administering to a subject with said disease an effective amount of a compound having the formula: 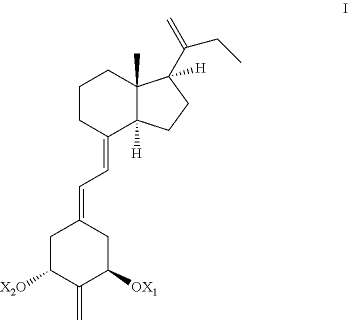
where X₁ and X₂, which may be the same or different, are each selected from hydrogen or a hydroxy-protecting group.

32. The method of claim 31 wherein the compound is administered orally.

33. The method of claim 31 wherein the compound is administered parenterally.

34. The method of claim 31 wherein the compound is administered transdermally.

35. The method of claim 31 wherein the compound is administered rectally

36. The method of claim 31 wherein the compound is administered nasally.

37. The method of claim 31 wherein the compound is administered sublingually.

38. The method of claim 31 wherein the compound is administered in a dosage of from about 0.01 μg/day to about 1000 μg/day.

39. The method of claim 31 wherein the compound has the formula: 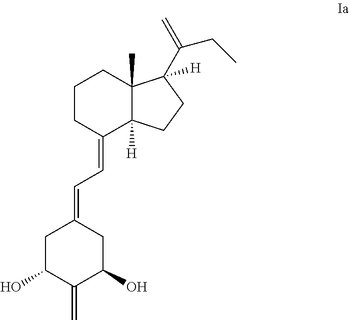 [SMILES: C=C1[C@H](O)CC(=C/C=C2\CCC[C@]3(C)[C@@H](C(=C)CC)CC[C@@H]23)C[C@H]1O]
and is named 2-methylene-20(21)-dehydro-19,24,25,26,27-pentanor-1α-hydroxyvitamin D₃.

40. A method of treating an inflammatory disease selected from the group consisting of rheumatoid arthritis, asthma, and inflammatory bowel diseases, comprising administering to a subject with said disease an effective amount of a compound having the formula: 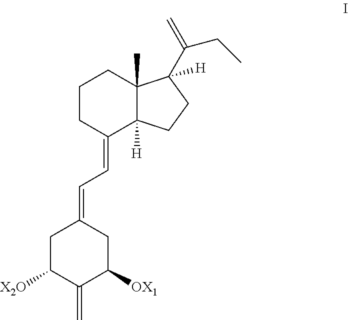
where X₁ and X₂, which may be the same or different, are each selected from hydrogen or a hydroxy-protecting group.

41. The method of claim 40 wherein the compound is administered orally.

42. The method of claim 40 wherein the compound is administered parenterally.

43. The method of claim 40 wherein the compound is administered transdermally.

44. The method of claim 40 wherein the compound is administered rectally.

45. The method of claim 40 wherein the compound is administered nasally.

46. The method of claim 40 wherein the compound is administered sublingually.

47. The method of claim 40 wherein the compound is administered in a dosage of from about 0.01 μg/day to about 1000 μg/day.

48. The method of claim 40 wherein the compound has the formula: 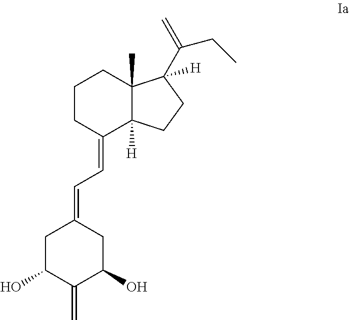 [SMILES: C=C1[C@H](O)CC(=C/C=C2\CCC[C@]3(C)[C@@H](C(=C)CC)CC[C@@H]23)C[C@H]1O]
and is named 2-methylene-20(21)-dehydro-19,24,25,26,27-pentanor-1α-hydroxyvitamin D₃.

49. A method of treating a skin condition selected from the group consisting of wrinkles, lack of adequate skin firmness, lack of adequate dermal hydration and insufficient sebum secretion which comprises administering to a subject with said skin condition an effective amount of a compound having the formula: 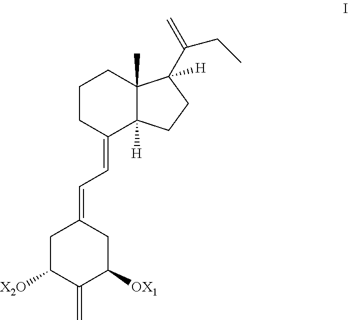
where X₁ and X₂, which may be the same or different, are each selected from hydrogen or a hydroxy-protecting group.

50. The method of claim 49 wherein the compound is administered orally.

51. The method of claim 49 wherein the compound is administered parenterally.

52. The method of claim 49 wherein the compound is administered transdermally.

53. The method of claim 49 wherein the compound is administered topically.

54. The method of claim 49 wherein the compound is administered rectally.

55. The method of claim 49 wherein the compound is administered nasally.

56. The method of claim 49 wherein the compound is administered sublingually.

57. The method of claim 49 wherein the compound is administered in a dosage of from about 0.01 μg/day to about 1000 μg/day.

58. The method of claim 49 wherein the compound has the formula: 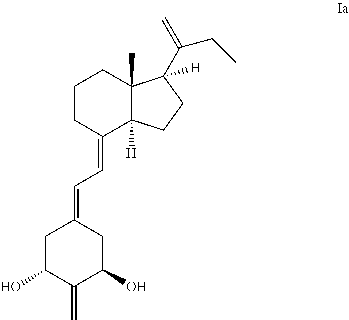 [SMILES: C=C1[C@H](O)CC(=C/C=C2\CCC[C@]3(C)[C@@H](C(=C)CC)CC[C@@H]23)C[C@H]1O]
and is named 2-methylene-20(21)-dehydro-19,24,25,26,27-pentanor-1α-hydroxyvitamin D₃.

59. A method of treating renal osteodystrophy comprising administering to a subject with renal osteodystrophy an effective amount of a compound having the formula: 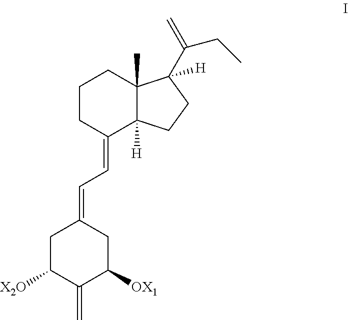
where X₁ and X₂, which may be the same or different, are each selected from hydrogen or a hydroxy-protecting group.

60. The method of claim 59 wherein the compound is administered orally.

61. The method of claim 59 wherein the compound is administered parenterally.

62. The method of claim 59 wherein the compound is administered transdermally.

63. The method of claim 59 wherein the compound is administered rectally.

64. The method of claim 59 wherein the compound is administered nasally.

65. The method of claim 59 wherein the compound is administered sublingually.

66. The method of claim 59 wherein the compound is administered in a dosage of from about 0.01 μg/day to about 1000 μg/day.

67. The method of claim 59 wherein the compound has the formula: 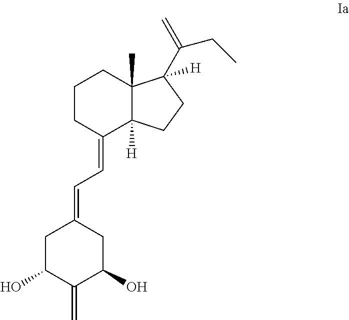 [SMILES: C=C1[C@H](O)CC(=C/C=C2\CCC[C@]3(C)[C@@H](C(=C)CC)CC[C@@H]23)C[C@H]1O]
and is named 2-methylene-20(21)-dehydro-19,24,25,26,27-pentanor-1α-hydroxyvitamin D₃.

68. A method of treating or preventing obesity of an animal, inhibiting adipocyte differentiation, inhibiting SCD-1 gene transcription, and/or reducing body fat in an animal comprising administering to an animal in need thereof an effective amount of a compound having the formula: 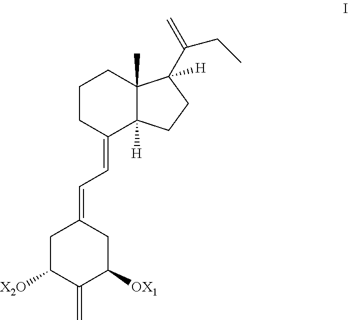
where X₁ and X₂, which may be the same or different, are each selected from hydrogen or a hydroxy-protecting group.

69. The method of claim 68 wherein the compound is administered orally.

70. The method of claim 68 wherein the compound is administered parenterally.

71. The method of claim 68 wherein the compound is administered transdermally.

72. The method of claim 68 wherein the compound is administered rectally.

73. The method of claim 68 wherein the compound is administered nasally.

74. The method of claim 68 wherein the compound is administered sublingually.

75. The method of claim 68 wherein the compound is administered in a dosage of from about 0.01 μg/day to about 1000 μg/day.

76. The method of claim 68 wherein the compound has the formula: 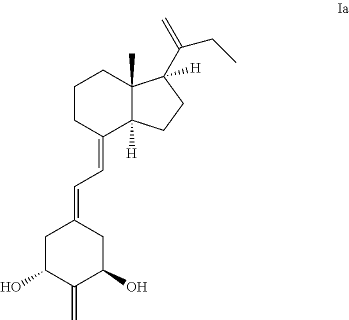 [SMILES: C=C1[C@H](O)CC(=C/C=C2\CCC[C@]3(C)[C@@H](C(=C)CC)CC[C@@H]23)C[C@H]1O]
and is named 2-methylene-20(21)-dehydro-19,24,25,26,27-pentanor-1α-hydroxyvitamin D₃.

77. The method of claim 68 wherein the animal is a human.

78. The method of claim 68 wherein the animal is a domestic animal.

79. The method of claim 68 wherein the animal is an agricultural animal.

80. A method of treating secondary hyperparathyroidism of renal osteodystrophy comprising administering to a subject with secondary hyperparathyroidism of renal osteodystrophy an effective amount of a compound having the formula: 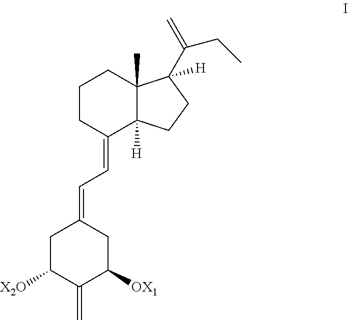
where X₁ and X₂, which may be the same or different, are each selected from hydrogen or a hydroxy-protecting group.

81. The method of claim 80 wherein the compound is administered orally.

82. The method of claim 80 wherein the compound is administered parenterally.

83. The method of claim 80 wherein the compound is administered transdermally.

84. The method of claim 80 wherein the compound is administered rectally.

85. The method of claim 80 wherein the compound is administered nasally.

86. The method of claim 80 wherein the compound is administered sublingually.

87. The method of claim 80 wherein the compound is administered in a dosage of from about 0.01 μg/day to about 1000 μg/day.

88. The method of claim 80 wherein the compound has the formula: 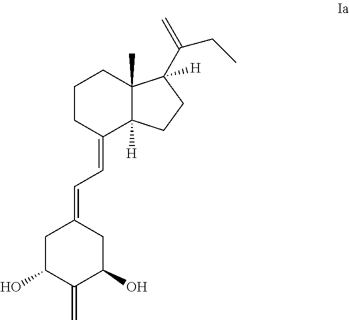 [SMILES: C=C1[C@H](O)CC(=C/C=C2\CCC[C@]3(C)[C@@H](C(=C)CC)CC[C@@H]23)C[C@H]1O]
and is named 2-methylene-20(21)-dehydro-19,24,25,26,27-pentanor-1α-hydroxyvitamin D₃.
